(19) United States
(12) Patent Application Publication
(10) Pub. No.: US 2005/0250114 A1
(43) Pub. Date: Nov. 10, 2005

(54) Method for decreasing the number of free virus particles within the bodily fluids of a virally-infected mammal

(76) Inventor: Essam T. Awdalla, Raleigh, NC (US)

Correspondence Address: Essam T. Awdalla, 2309 Myron Drive, Apt. F, Raleigh, NC, 27607, US

(21) Appl. No.: 10/841,746

(22) Filed: May 7, 2004

(51) Int. Cl. C12Q 1/68; C12N 7/00; C12N 7/01; A61K 31/19; A01N 37/10

(52) U.S. Cl. 435/6; 435/235.1

(57) ABSTRACT

The present invention provides a method for decreasing the number of free virus particles within the bodily fluids of a mammal infected by a virus, comprising the steps of: a) specifying the type of the pathogenic virus; b) providing a supply of host cells susceptible to the specified virus type; c) treating the host cells provided in step (b) with one, or more than one viral replication inhibiting means; d) washing the host cells prepared in step (c) with a sterile physiological medium; and e) administering the host cells prepared in step (d) to the mammal. The host cells are administered to the mammal either by intravenous injection, injection into the CSF, or spraying within its respiratory tract passages.

### FIELD OF THE INVENTION

The present invention relates to a method used for decreasing the number of free virus particles within the bodily fluids of a virally-infected mammal, and thus attenuating the virally-induced pathogenic process.

### BACKGROUND OF THE INVENTION

Viruses are genetic elements containing either DNA or RNA. During their life cycle, viruses alternate between two distinct states, extracellular and intracellular. In the extra-cellular state a virus is a metabolically inert particle containing nucleic acids surrounded by proteins, and occasionally containing other components. Each virus particle has one or more proteins on its outer surface referred to as the “anti-receptor”, which interacts specifically with a certain cell surface component referred to as the “receptor” displayed on the outer surface of a certain type of cells referred to as “susceptible host cells.”

Once a virus particle gets in contact with its susceptible host cell, it attaches to it. The attached virus particle triggers the host cell to engulf it, or it fuses itself to the membrane of the host cell so it can release its genetic component into the cell. Once inside the cell, the genetic component of the virus overrides the host cell and uses it to replicate all the virus components. Finally, the mature virus particles are released from the host cell, and are hereinafter referred to as “free virus particles.”

In mammals, the consequences of a pathogenic viral infection depend upon a number of factors including the number of infecting viral particles and their path to susceptible host cells, the speed of viral replication and spread, the effect of the virus on cell functions, the host's secondary responses to the cellular injury, and the immunologic and non-specific defenses of the host. In general, virally-induced diseases are either acute, self limiting, infections or chronic, long term, infections.

In acute, self limiting, infections, the immune system of the mammal recognizes the virally-infected cells and the extracellular free virus particles, and launches a curative immune response against them. However, during the period needed for the mammal's immune system to recognize the pathogen in order to mount an effective immune response against it, considerable number of susceptible host cells would have already been infected by viruses, which will eventually be destroyed once the immune response is launched. So, there arises a need for decreasing the number of free virus particles within the bodily fluids of the mammal during this period to attenuate the signs, symptoms and probable complications associated with the acute stage of the disease, decrease the number of infected, eventually destroyed, host cells, and decrease the burden on the immune system of the infected mammal.

In chronic, long term, infections, the immune system of the mammal is usually incapable of launching a curative immune response against the virus. And as the growth of the virus is intimately tied to the host cell functions, so, it is difficult to specifically attack a virus by a chemotherapeutic agent without causing at least some harm to its hosting cell. So, there also arises a need for decreasing the number of free virus particles within the bodily fluids of the mammal during the initial acute stages of the diseases, as well as during the acute relapses, as an adjuvant to other used chemotherapeutic agents, in order to improve the mammal's long term prognosis.

### SUMMARY OF THE INVENTION

The present invention provides a method for decreasing the number of free virus particles within the bodily fluids of a mammal infected by a virus, without cross-reacting with any simultaneously administered anti-viral chemotherapeutic means, and without interfering with the development of an immune response against the virus.

Accordingly, the method provided in the present invention comprises the steps of:
a) specifying the type of the pathogenic virus;
b) providing a supply of host cells susceptible to the specified virus type;
c) treating the host cells provided in step (b) with one, or more than one, viral replication inhibiting means;
d) washing the host cells prepared in step (c) with a sterile physiological medium; and
e) administering the host cells prepared in step (d) to the mammal.

The host cells prepared in step (d) are administered to the mammal by intravenous injection, injection into the cerebrospinal fluid (CSF), or spraying within the respiratory tract passages. The administered host cells attract the free virus particles from the bodily fluids of the mammal and engulf them, but as these host cells were treated by viral replication inhibition means (step (c)), so, no replication of the virus particles occurs within them. And thus, virus particles will accumulate within the administered host cells, resulting in a marked decrease in the number of free virus particles within the mammal's bodily fluids.

These and other aspects of the present invention will become apparent to those skilled in the art after a reading of the following description of the preferred embodiment of the invention.

### DESCRIPTION OF THE INVENTION

The present invention provides a method for decreasing the number of free virus particles within the bodily fluids of a mammal infected by a pathogenic virus, comprising the steps of:

a) Specifying the Type of the Pathogenic Virus:

The type of the pathogenic virus is specified using either the direct or indirect ELIZA (Enzyme-linked immunosorbent assay) techniques. The direct ELIZA technique enables the specification of the type of the virus particles present in the blood, excreta, or bodily fluids of the infected mammal very early after the infection. The indirect ELIZA technique enables the specification of the type of the pathogenic virus indirectly, by identifying the type of antibodies developed against it by the infected mammal. These techniques are widely in use in clinical and research laboratories, and are well known to people experienced in the art.

b) Providing a Supply of Host Cells Susceptible to the Specified Virus Type:

Once the type of the pathogenic virus is specified, its susceptible host cell line(s) is/are determined. Such susceptible host lines, their culturing growth media, and their culturing vessels and surfaces are commercially available in the market from several suppliers such as the American Type Culture Collection (Manassas, Va.). Techniques used for cell culturing and harvesting depends on the type of cultured cell. Such techniques are well known to people experienced in the art.

c) Treating the Harvested Host Cells Provided in Step (b) with One, or More than One, Viral Replication Inhibiting Means:

As used herein, the “viral replication inhibiting means” refers to a chemotherapeutic agent known to inhibit the replication of viruses by disabling one of its replication stages. Several of these chemotherapeutics are already in use, or being developed. Non-limiting examples of in vivo used viral replication inhibiting means are the Alpha interferon, Ribavirin (Virazole), and Azidothymidine (AZT, Zidovudine, Retrovir), and equivalents thereof. Some other chemotherapeutic agents known to have potent viral replication inhibiting effect cannot be used in vivo, because of their lethal effect on the normal cells of the mammal, and thus their use is restricted for in vitro research purposes. Non-limiting examples of in vitro viral replicating inhibiting means are the Diphtheria toxin and the amanitin, and equivalents thereof. These and other used viral replication inhibiting means are widely in use, and well known to people experienced in the art.

Although any viral replication inhibiting means, or a combination thereof, may be used, yet it is preferable to use at least one of the protein synthesis inhibitors as a viral replication inhibiting means, as protein synthesis inhibitors will also inhibit the secretion of Interferon by the administered host cells, which, if secreted, will interfere with the free admission of virus particles to the administered host cells. The use of protein synthesis inhibitors also safeguards against any uncontrolled multiplication of the host cells after their administration to the mammal.

Non-limiting examples of protein synthesis inhibitors include the Diphtheria toxin, the Alpha interferon, and the protein synthesis inhibitor GLQ223 previously tested by Genelabs (Redwood City, Calif.), and equivalents thereof.

d) Washing the Host Cells Prepared in Step (c) with a Sterile Physiological Medium:

The treated host cells are repeatedly washed from the used viral replication inhibiting means by a physiological medium. As used herein, a physiological medium is a solution having precise amounts of organic and/or inorganic components, within which the integrity of the host cells is maintained.

For example, using strictly aseptic techniques, the host cells are repeatedly washed in Ringer's solution followed by separation of the cells from the formed suspension by centrifugation at relatively low speeds (around 10,000×g for 10 minutes). Such procedures are well known by people experienced in the art.

e) Administering the Host Cells Prepared in Step (d) to the Mammal:

The route(s) of administration of the host cells prepared in step (d) to the mammal is determined according to the route within which the target free virus particles are known to spread within the body of the mammal. So, for virus particles known to spread through the blood, the host cells are administered by intravenous injection, which is preceded by testing the mammal for sensitivity against the used type of host cells, to avoid the development of any adverse reactions. For virus particles known to spread through the respiratory tract passages, the host cells are administered by spraying within the respiratory tract passages of the mammal. Direct injection of the host cells to the cerebrospinal fluid (CSF) is used in cases of viral encephalitis and meningitis, as the virus particles will be most abundant within the CSF.

Either freshly prepared host cells are administered, or, whenever needed, the prepared host cells are temporarily stored using well known Cryopreservation techniques, and in this case, the host cells are warmed up before their administration to the mammal.

As described hereinbefore, the administered host cells attract the free virus particles from the bodily fluids (serum of the blood, CSF, or secretions within the respiratory tract passages) of the mammal and engulf them, but as the host cells were treated by viral replication inhibition means (step (c)), so, no replication of the virus particles occurs within the administered host cells. And thus, virus particles will accumulate within the administered host cells, resulting into marked decrease in the number of free virus particles within the mammal's bodily fluids. Eventually, the administered host cells will be destroyed, either after being detected by the mammal's immune system as non-self, or due to normal aging processes taking place within them. In both cases, the released virus particle fragments will be inactive, due to the disarrangement between the virus particle coating proteins and its genetic component, which occurs during the entry of the virus particle to the host cell.

Certain modifications and improvements will occur to those skilled in the art upon a reading of the foregoing description. All modifications and improvements have been deleted herein for the sake of conciseness and readability but are properly within the scope of the following claims.

1. In a mammal infected by a pathogenic virus,
a method for decreasing the number of free virus particles within the bodily fluids of the said mammal, comprising the steps of:
a) specifying the type of the pathogenic virus;
b) providing a supply of host cells susceptible to the specified virus type;
c) treating the host cells provided in step (b) with viral replication inhibiting means;
d) washing the host cells prepared in step (c) with a sterile physiological medium; and
e) administering the host cells prepared in step (d) to the mammal.

2. The method of claim 1, wherein step (e) further comprises temporary Cryopreservation of the host cells prepared in step (d) prior to their administration to the mammal.

3. The method of claim 1, wherein the host cells prepared in step (d) are administered to the mammal by intravenous injection.

4. The method of claim 1, wherein the host cells prepared in step (d) are administered to the mammal by spraying within the respiratory tract passages.

5. The method of claim 1, wherein the host cells prepared in step (d) are administered to the mammal by injecting into the CSF.

6. The method of claim 1, wherein said viral replication inhibiting means is a protein synthesis inhibitor.

7. The method of claim 1, wherein the mammal is a man.

8. The method of claim 1, wherein the step of administering the host cells results in a decrease of the number of free virus particles within the bodily fluids of the mammal.

9. The method of claim 8, wherein the decrease in the number of free virus particles occurs without cross-reacting with any simultaneously administered antiviral chemotherapy drugs.

10. The method of claim 8, wherein the decrease in the number of free virus particles occurs without interfering with the immune response of the mammal.

11. In a mammal infected by a pathogenic virus,
a method for decreasing the number of free virus particles within the bodily fluids of the said mammal, comprising the steps of:
a) specifying the type of the pathogenic virus;
b) providing a supply of host cells susceptible to the specified virus type;
c) treating the host cells provided in step (b) with more than one viral replication inhibiting means;
d) washing the host cells prepared in step (c) with a sterile physiological medium; and
e) administering the host cells prepared in step (d) to the mammal.

12. The method of claim 11, wherein step (e) further comprises temporary Cryopreservation of the host cells prepared in step (d) prior to their administration to the mammal.

13. The method of claim 11, wherein the host cells prepared in step (d) are administered to the mammal by intravenous injection.

14. The method of claim 11, wherein the host cells prepared in step (d) are administered to the mammal by spraying within the respiratory tract passages.

15. The method of claim 11, wherein the host cells prepared in step (d) are administered to the mammal by injecting into the CSF.

16. The method of claim 11, wherein at least one of the said viral replication inhibiting means is a protein synthesis inhibitor.

17. The method of claim 11, wherein the mammal is a man.

18. The method of claim 11, wherein the step of administering the host cells results in a decrease of the number of free virus particles within the bodily fluids of the mammal.

19. The method of claim 18, wherein the decrease in the number of free virus particles occurs without cross-reacting with any simultaneously administered antiviral chemotherapy drugs.

20. The method of claim 18, wherein the decrease in the number of free virus particles occurs without interfering with the immune response of the mammal.
